(19) United States
(12) Patent Application Publication
(10) Pub. No.: US 2012/0295248 A1
(43) Pub. Date: Nov. 22, 2012

(54) SYSTEMS AND METHODS FOR DYNAMIC GAS CONTROL IN A DISPOSABLE VESSEL

(76) Inventor: Alan T. Cheng, Naperville, IL (US)

(21) Appl. No.: 13/475,294

(22) Filed: May 18, 2012

Related U.S. Application Data
(60) Provisional application No. 61/487,806, filed on May 19, 2011.

(51) Int. Cl. C12Q 3/00; C12M 1/36

(52) U.S. Cl. 435/3; 435/286.1; 435/286.6

(57) ABSTRACT

A disposable mixing vessel incorporates a system for dynamic gas control. The disposable mixing vessel includes a first agitating element and a second agitating element located above the first agitating element. The system for dynamic gas control enables a cell culture process to occur in the disposable mixing vessel while maintaining certain parameters of the liquid medium to be within a desired range. Such parameters may include the level of dissolved carbon dioxide, the level of dissolved oxygen, the pH and the osmolality. The system for dynamic gas control may control gas inlet and outlet ports in the disposable mixing vessel for sweeping a suitable composition of gas across the headspace of the liquid medium.

### CROSS REFERENCE TO RELATED APPLICATIONS

The present application claims priority from U.S. provisional patent application Ser. No. 61/487,806 filed May 19, 2011.

### FIELD OF THE INVENTION

Aspects relate to systems and methods for controlling properties of a liquid within a disposable vessel. For example, properties such as levels of dissolved carbon dioxide, dissolved oxygen, pH and osmolality within a liquid medium may be dynamically controlled in a biomass culture process.

### BACKGROUND

As cells are cultured for a variety of applications, many have sought to optimize processes for cell growth in a liquid medium. For example, certain parameters of the medium in which cells are grown are important for cultivating healthy cells, such as pH, level of dissolved oxygen, level of dissolved carbon dioxide, temperature and nutrient composition. In addition, processing patterns for growing cells are also considered, such as how the medium may be mixed, what methods of gas input/exhaust may be used or how nutrients are added to the medium. A seed culture inoculum is typically prepared prior to large scale manufacturing processes for cultivating mammalian cells in a bioreactor. Cells are cultivated in a series of incubators and/or small bioreactors of increasing volume until a sufficient volume and density of cells is available as a seed culture for inoculation into a larger production bioreactor. Once the seed culture is inoculated into the bioreactor medium, parameters such as pH, temperature, level of dissolved carbon dioxide, osmolality and level of dissolved oxygen may be monitored.

In a cell culture medium, desired levels of dissolved oxygen are typically obtained through air sparging using a sparger installed at the bottom of the bioreactor while agitating the culture medium using impellers. Impeller agitation serves to distribute gas and liquid to enhance oxygen transfer within the cell medium. However, during the cell growth process, levels of dissolved carbon dioxide and/or osmolality within the bioreactor may also rise due to contributions from chemical and/or biological sources. For example, when the culture medium is slightly alkaline, carbon dioxide may be sparged directly into the culture medium for reducing pH to a prescribed level, usually around 7.0. Carbon dioxide is also a product of respiration from mammalian cells contained within the bioreactor. Though, as more carbon dioxide is produced, the pH of the cell culture medium becomes more acidic, oftentimes requiring a bicarbonate buffer solution to be added for maintaining a desired range of pH in the cell culture medium. Such buffer solutions containing sodium bicarbonate and/or sodium carbonate may contribute as a chemical source of osmolality in the medium.

### SUMMARY OF THE INVENTION

Aspects described herein relate to processing a liquid contained within a disposable mixing vessel. In some embodiments, the disposable mixing vessel may be a disposable bioreactor or a disposable fermentor. The disposable mixing vessel includes a first agitating element located at a lower mixing region of the disposable mixing vessel and a second agitating element located above the first agitating element. Installed within the disposable mixing vessel is a system for dynamically controlling gas dissolved within the liquid as well as maintaining certain attributes of the liquid. A first volume of liquid containing living organisms (e.g., cells) is added to the disposable mixing vessel where the liquid has a top surface level that reaches above the first agitating element, yet remains below the second agitating element. The first agitating element is then operated to mix the first volume of liquid so as to provide a suitable living and growth environment for the living organisms contained in the liquid. Subsequent to operating the first agitating element in the manner described, a second volume of liquid is added to the first volume such that the combined volume of liquid has a top surface level that reaches above or is in contact with the second agitating element. The second agitating element is then operated to mix the combined volume of liquid. In continuing to provide a suitable living environment for the organisms contained within the liquid, the system for dynamic gas control is utilized in conjunction with the second agitating element to maintain at least one of a range of dissolved carbon dioxide, a range of dissolved oxygen, a range of pH or a range of osmolality within the liquid.

Implementing the system for dynamic gas control in the disposable mixing vessel may involve incorporating a number of processing components. For example, the disposable mixing vessel may include a gas inlet port and a gas outlet port for sweeping gas through a headspace above the top surface of the liquid. The disposable mixing vessel may also include a number of sensors for sensing any number of parameters regarding the liquid, such as levels of dissolved carbon dioxide, levels of dissolved oxygen, pH or osmolality. In various embodiments, the second agitating element produces currents within the liquid that induce flow in a generally upward direction within the disposable mixing vessel. A controller in connection with the disposable mixing vessel may be configured to control operation of any components provided with the disposable mixing vessel, such as agitating elements, gas inlet port(s), gas outlet port(s) and/or sensor(s).

The controller of the system for dynamic gas control of liquid within the disposable mixing vessel may be configured to operate in two modes. The first mode involves operation of components within the disposable mixing vessel when a top surface of the liquid in the vessel is disposed between the first and second agitating elements. The second mode involves operation of components within the disposable mixing vessel when both agitating elements are either fully or partially submerged under the liquid within the vessel. While operating in the first mode, the first agitating element is activated to mix the liquid in the vessel at a speed suitable for growing living organisms. Other appropriate processing components within the disposable mixing vessel may also be activated, such as suitable gas inlet/outlet ports and/or conduits for adding nutrients. In the second mode, based on one of the sensed parameters of the dissolved carbon dioxide, dissolved oxygen, pH or osmolality of the liquid, the speed at which the second agitating element is operated and/or a characteristic of gas flow through the headspace above the top surface of the liquid are controlled so as to maintain levels of dissolved carbon dioxide, dissolved oxygen, pH or osmolality within the liquid to be within desired ranges.

In an illustrative embodiment, a method of processing a liquid is provided. The method includes providing a disposable mixing vessel having a first agitating element located at a lower mixing region of the disposable mixing vessel; providing a system for dynamic gas control within the disposable mixing vessel employing a second agitating element located above the first agitating element; adding a first volume of liquid to the disposable mixing vessel having a top surface height that is lower than the second agitating element within the disposable mixing vessel; providing living organisms in the first volume of liquid; operating the first agitating element to mix the first volume of liquid within the disposable mixing vessel and to help provide a suitable living environment for the living organisms; subsequent to operating the first agitating element, adding a second volume of liquid to the first volume of liquid within the disposable mixing vessel resulting in a combined volume of liquid that has a top surface height that is higher than the second agitating element; operating the second agitating element to mix the combined volume of liquid within the disposable mixing vessel and to help provide a suitable living environment for the living organisms; and operating the system for dynamic gas control for maintaining at least one of a range of dissolved carbon dioxide, a range of dissolved oxygen, a range of pH or a range of osmolality within the combined volume of liquid.

In another illustrative embodiment, a system for processing a liquid is provided. The system includes a disposable mixing vessel constructed and arranged to contain the liquid; a first agitating element located at a lower mixing region of the disposable mixing vessel; a second agitating element located above the first agitating element and constructed and arranged to direct flow of liquid upward within the disposable mixing vessel; a gas inlet port disposed at an upper region of the disposable mixing vessel adapted to provide entry of a gas into a headspace above the top surface of the liquid; a gas outlet port disposed at the upper region of the disposable mixing vessel adapted to permit exit of the gas from the headspace above the top surface of the liquid; at least one sensor for sensing at least one of a level of dissolved carbon dioxide, a level of dissolved oxygen, a pH or a level of osmolality of the liquid; and a controller configured to control operation of the first and second agitating elements, the gas inlet and outlet ports and the at least one sensor in two operating modes including a first mode in which a liquid level in the vessel is above the first agitating element and below the second agitating element, and a second mode in which the liquid level in the vessel is above the second agitating element, the first mode including controlling the first agitating element to mix the liquid in the tank at a speed suitable for living organisms in a condition with the liquid level below the second agitating element, and the second mode including controlling a speed of the second agitating element or a characteristic of gas flow through the headspace above the top surface of the liquid based on a sensed parameter of the at least one of the level of dissolved carbon dioxide, the level of dissolved oxygen, the pH or the level of osmolality of the liquid so as to maintain at least one of a range of dissolved carbon dioxide, a range of dissolved oxygen, a range of pH or a range of osmolality within the liquid.

Further features and advantages of the present invention, as well as the structure of various embodiments of the present invention are described in detail below with reference to the accompanying drawings.

### BRIEF DESCRIPTION OF DRAWINGS

The accompanying drawings are not intended to be drawn to scale. In the drawings, identical or nearly identical components that are illustrated in various figures are represented by like numerals. For purposes of clarity, not every component may be labeled in every drawing. In the drawings:

FIG. 1 is a schematic representation of an embodiment of a disposable mixing vessel;

FIG. 2 is a schematic representation of the embodiment of FIG. 1 where liquid is added to the disposable mixing vessel;

FIG. 3 is a schematic representation of the embodiment of FIGS. 1 and 2 where more liquid is added to the disposable mixing vessel; and

FIG. 4 is a flowchart of process steps for operating a system for dynamic gas control within a disposable mixing vessel.

### DETAILED DESCRIPTION

In embodiments described herein, incorporating a system for dynamic gas control in a disposable mixing vessel, such as a disposable bioreactor or a disposable fermentor, may be advantageous for certain chemical/biological processes. Liquids disposed within such vessels may include a suitable number of living organisms, such as bacteria or mammalian cells. Cells contained within a disposable mixing vessel may be subject to a seed-train inoculation process where cell lines are incubated and subsequently expanded for large scale production. The disposable mixing vessel includes at least a first agitating element located at a lower mixing region of the disposable mixing vessel and at least a second agitating element located above the first agitating element. A system for dynamic gas control is installed with the disposable mixing vessel so that certain parameters of the liquid may be maintained, providing a suitable growth environment for the organisms. The system for dynamic gas control may be incorporated with an appropriate process for culturing cells or other organisms within the disposable mixing vessel.

In an embodiment, a liquid medium containing living cells is added to the disposable mixing vessel such that the volume of the liquid medium reaches above the first agitating element yet remains below the second agitating element. The first agitating element is operated to mix the liquid medium in manner that facilitates cell growth. That is, currents generated by the first agitating element are of sufficient strength to distribute much needed nutrients and gases to the cells, yet are not excessively strong to the point where cells are damaged or destroyed. Subsequent to mixing the first volume of liquid medium, more liquid medium having cells contained therein is added to the disposable mixing vessel so as to fully or partially cover both the first and second agitating elements. The second agitating element is then employed to mix the liquid medium in conjunction with operation of the system for dynamic gas control so as to maintain at least one of a range of dissolved carbon dioxide, a range of dissolved oxygen, a range of pH or a range of osmolality within the liquid. At any suitable point during the process of cell culture, any appropriate nutrient(s) and/or gas(es) may be added to the liquid medium via a processing component coupled with the disposable mixing vessel.

The disposable mixing vessel may include any number of processing components that function in association with the system for dynamic gas control. In some embodiments, the disposable mixing vessel includes a gas inlet port and a gas outlet port for sweeping gas through a headspace above the top surface of the liquid medium. The disposable mixing vessel may also include any suitable number and type of sensor(s) for sensing any appropriate parameter(s) regarding the liquid, such as levels of dissolved carbon dioxide, levels of dissolved oxygen, pH or osmolality. In some cases, the second agitating element generates currents within the liquid medium that direct flow of liquid upward within the disposable mixing vessel toward the top surface of the liquid. The system for dynamic gas control may include a controller associated with the disposable mixing vessel which may be configured to monitor, based on information gathered from the sensor(s), and control operation of any suitable processing component(s) provided with the disposable mixing vessel.

The system for dynamic gas control may be configured to control processing components of the disposable mixing vessel in at least two operating modes. When the level of the top surface of the liquid medium in the disposable mixing vessel is above the first agitating element yet below the second agitating element, the first operating mode for the system for dynamic gas control may be triggered. In the first operating mode of the system for dynamic gas control, the first agitating element produces currents in the liquid medium that mix the liquid in the vessel in a manner that creates a suitable growth environment for cells contained within the liquid medium. In some embodiments, operating the first agitating element while in the first mode involves running the first agitating element at a generally low velocity so as to gently mix the liquid medium without incurring damage to the cells.

The second operating mode for the system for dynamic gas control may be activated when additional liquid medium is added to the disposable mixing vessel such that the level of liquid medium rises above both the first and second agitating elements. When more liquid medium is added to the disposable mixing vessel, the first and/or second agitating element(s) may be operated at a velocity higher than that when there was less liquid medium contained within the vessel. Increasing agitating velocities may be suitable after an initial cell proliferation period, so that contents within the disposable mixing vessel, being more plentiful within the vessel, may be more evenly mixed. In the second operating mode, the velocity at which the second agitating element is operated and/or a characteristic of gas flow through the headspace above the top surface of the liquid medium may be adjusted based on one or more of the sensed parameters of the dissolved carbon dioxide, dissolved oxygen, pH or osmolality of the liquid. For example, if the system determines that the level of dissolved carbon dioxide in the liquid is too high, then the velocity at which the second agitating element operates may increase and/or the rate of gas flow through the headspace of the liquid medium may also increase. Such adjustments would effectively provide for an increasing upward flow of liquid medium containing dissolved carbon dioxide toward the surface while also inducing carbon dioxide to come out of solution at the gas-liquid interface and join carbon dioxide gas flow through the headspace. Accordingly, the adjustments set forth by the system for dynamic gas control provides for levels of dissolved carbon dioxide, dissolved oxygen, pH or osmolality within the liquid to be maintained within desired ranges.

System and methods for dynamic gas control in industrial bioreactors like those generally described in U.S. Patent Publication No. 2010/0035343 entitled “System and Method for Controlling a Mammalian Cell Culture Process”; U.S. Patent Publication No. 2010/0184147 entitled “Method for Controlling pH, Osmolality and Dissolved Carbon Dioxide Levels in a Mammalian Cell Culture Process to Enhance Cell Viability and Biologic Product Yield”; and U.S. Patent Publication No. 2010/0035342 entitled “Method for Controlling pH, Osmolality and Dissolved Carbon Dioxide Levels in a Mammalian Cell Culture Process to Enhance Cell Viability and Biologic Product Yield” may be used for dynamically controlling gas in accordance with aspects of embodiments described herein.

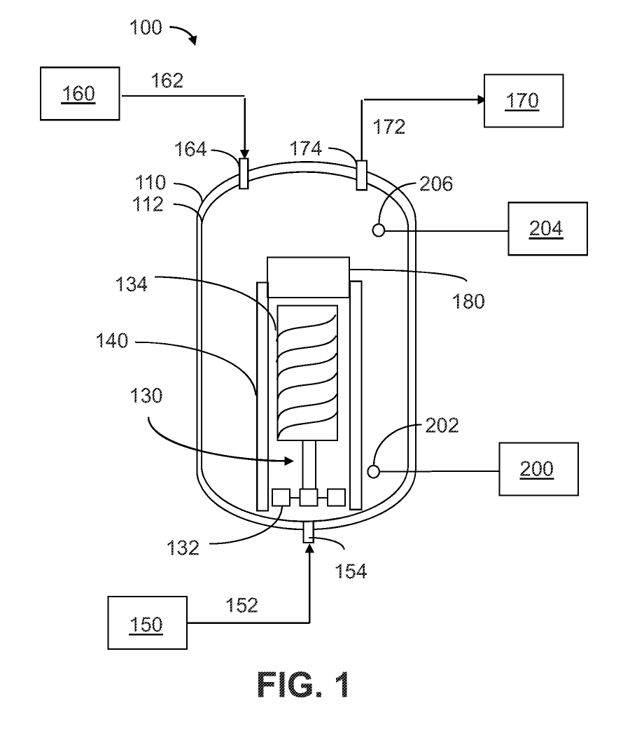

FIG. 1 depicts an illustrative embodiment of a system 100 where a disposable mixing vessel 112 is installed within a support tank 110. In some embodiments, as described above, the disposable mixing vessel 112 may be a disposable bioreactor or a disposable fermentor. Accordingly, the disposable mixing vessel 112 may include a thin plastic bag that lines the walls of the support tank 110. The disposable mixing vessel may also have a number of processing components that are useful for running a reaction and/or creating a suitable environment for organisms, such as cells, to grow and proliferate. Any suitable component(s) may be provided with the disposable mixing vessel 112, for example, biological filters, a sterilizing system, gas ports, sensors, impellers, or the like.

Located inside the disposable mixing vessel 112 is an agitation system 130 that includes a first agitating element 132 and a second agitating element 134. The first agitating element 132 is disposed at a lower region of the disposable mixing vessel 112 below the second agitating element 134. The first and second agitating elements 132, 134 are disposed within a draft tube 140, which may be cylindrical in shape. The draft tube 140 functions effectively as a shield between the space occupied by the agitating elements 132, 134 and the outer space within the disposable mixing vessel surrounding the agitating elements 132, 134. Attached at an upper region of the draft tube 140 is a vertical baffle 180 that functions to translate radially or rotationally oriented flow from a liquid into a more vertically directed flow.

Agitation systems described may incorporate any appropriate configuration of agitating elements. Indeed, as illustrated, agitating elements included in an agitation system need not be the same. For example, in embodiments not explicitly illustrated, a Ruston turbine or a pitched blade turbine may be employed as a first agitating element. As the second agitating element may apply an upward pumping flow to the liquid, in some embodiments, the second agitating element includes a screw impeller, a helical impeller or propeller. For example, such an impeller may provide upward flow while minimizing lateral or radial flow from the impeller, and reducing shear and other such forces that may cause damage to cells in the liquid.

Any number of agitating elements may be used in an agitation system, for example, one, two, three, four, or even more agitating elements. Further, agitating elements may share the same shaft and, in some cases, are operated in concert. Though, it can be appreciated that suitable agitating elements can be operated independently, and indeed, may incorporate separate shafts, or might not incorporate a shaft at all. A suitable agitating element may include a paddle that moves back and forth without rotation, or alternatively, a transducer that emits sonic energy into a mixing region to direct fluid flow and/or cause mixing agitation. In some embodiments, an agitating element may have a surface that is constructed in a manner such that gas injected toward the agitating element collects behind the surface and is subsequently ejected from the center of the agitating element outward. Or, dimensions of agitating blades may be adjusted so as to generate a certain profile of currents) within the liquid. For example, an impeller blade may be tilted or moved so that currents produced by the impeller will produce more upward directed flow as compared to radial or vortex-type flow. In some embodiments, agitating elements may be coupled to and driven by a motor that is located exterior to the disposable mixing vessel.

In some embodiments, the second agitating element 134 may be an upward flow impeller that induces fluid flow in an upward direction during operation. As such, the second agitating element 134 may provide a method for continuously renewing a top surface of liquid medium within the disposable mixing vessel with liquid from other regions of the vessel (e.g., the center of the vessel), so as to enhance and control surface gas exchange between the liquid medium and the headspace of gas above the liquid medium.

In some embodiments, a disposable mixing vessel is constructed to include a single-use impeller affixed to a lower portion of a flexible plastic bag. The impeller may include an impeller hub mounted on to a post where the impeller hub has an impeller blade suitable for creating currents upon impeller rotation. The impeller hub may be coupled to the shaft of a motor that may be provided exterior to or within the support structure of the disposable mixing vessel. In some embodiments, the flexible plastic bag is adapted to be mounted within the support structure such that the motor comes into an aligned arrangement with the impeller hub so that the motor may drive the impeller hub as a shaft of the motor rotates. The flexible plastic bag may also have connection ports for coupling various processing components within the bag (e.g., sensors, inlets/outlets, etc.) to suitable devices that enable such components to function properly. Such devices may be located exterior to the bag. The flexible plastic bag may also include a plastic draft tube and one or more vertical baffles that may be discarded with the bag after use.

Once the process (e.g., cell culture) carried out within the disposable mixing vessel is completed, the contents of the disposable mixing vessel may be suitably removed and the disposable mixing vessel, along with its respective components, may be taken away to be disposed of as waste and/or recycled.

In some embodiments, components within a disposable mixing vessel may be coupled to devices exterior to the vessel via a suitable magnetic coupling arrangement. A magnetic coupling arrangement may provide for a convenient attachment between components within the disposable mixing vessel and outside devices that provide appropriate functionality to the components.

A suitable gas is supplied to the bottom of the disposable mixing vessel 112 through an inlet port 154. Gas source 150, external to the vessel, supplies air through a conduit 152, such as a pipe, leading to the inlet port 154 so as to introduce the gas into the disposable mixing vessel. Thus, when the disposable mixing vessel 112 includes a liquid medium, gas introduced through inlet port 154 is delivered directly into the liquid medium.

Gas may be supplied into an upper region of the disposable mixing vessel 112 from a gas source 160 via a conduit 162 and through inlet port 164. Conversely, gas may be removed from the upper region of the disposable mixing vessel 112 through an outlet port 174 through a conduit 172 and to a gas exhaust 170. Gas source 160 and gas exhaust 170 may be located exterior to the disposable mixing vessel 112. Oxygen, nitrogen, air, carbon dioxide or other gases and mixtures thereof may sweep across the headspace above a surface of a liquid medium through introduction and removal via inlet port 164 and outlet port 174.

Another processing component or set of processing components suitable for use in the disposable mixing vessel 112 includes a sensor system 200, 204 having one or more corresponding probes 202, 206. While not expressed depicted, sensor system 200, 204 may include any number of probes and/or sensing devices for monitoring various attributes of the contents within the disposable mixing vessel. For example, the sensor system 200, 204 may include sensors that provide for monitoring levels of dissolved oxygen, levels of dissolved carbon dioxide, pH, osmolality, temperature, pressure, cell density, cell size, concentration of nutrient(s) or other appropriate parameters of a liquid medium. The sensor system 200, 204 may also be equipped to provide monitoring of other characteristics within the disposable mixing vessel, such as the rate of gas flow within the headspace above the liquid medium, the content of the gas within the headspace and/or exhaust, or other useful parameters. Suitable sensor systems may be coupled with a controller configured to send control signals to other processing components or devices for operating such processing components based on information provided from the sensor system.

In some embodiments, a suitable system for dynamic gas control is installed in a conventional disposable mixing vessel. For example, the disposable mixing vessel may include a first impeller disposed at a lower region of the vessel, and the system for dynamic gas control, when installed in the disposable mixing vessel, may provide a second impeller suitable for operation in conjunction with dynamic gas control, the second impeller positioned at a location above the first impeller.

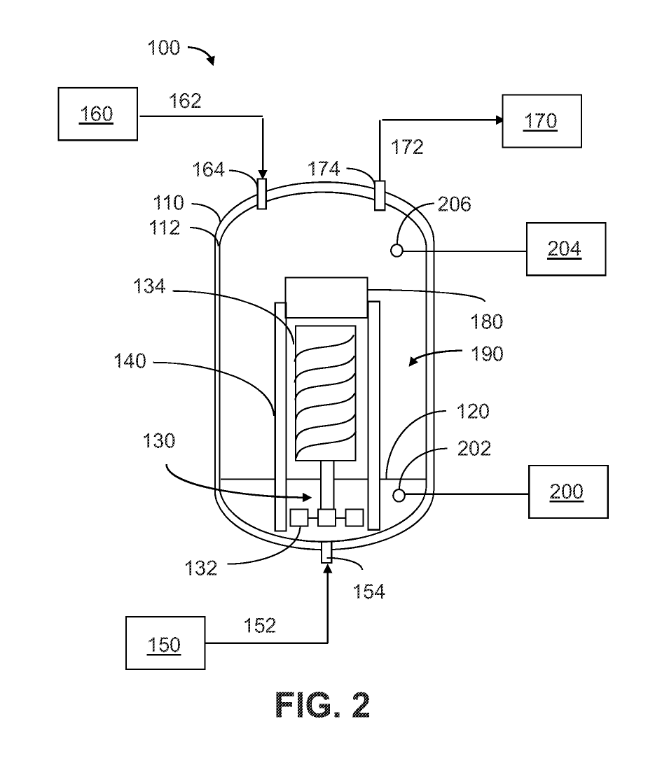

In FIG. 2, a first volume of liquid medium 120 is added to the disposable mixing vessel. The liquid medium includes a suitable density of cells along with nutrients and dissolved gas within the liquid for the cells to grow. As part of the seed-train process, the cells may be mixed with the first agitating element 132 at low speeds so as to cultivate cell proliferation. Sensor system 200 monitors parameters of the liquid medium 120, such as pH, temperature, dissolved oxygen, dissolved carbon dioxide, osmolality, concentration of nutrient(s) or other appropriate parameters using probe 202, and an assessment is made as to how viable the conditions of the liquid are for cells. Based on this assessment, parameters of the first agitating element may be adjusted to provide a greater or less degree of mixing. For example, if it is determined by a controller of the disposable mixing vessel that the liquid medium 120 does not have an even distribution of dissolved oxygen, leading to cells not having sufficient access to oxygen to grow, then the controller may send a signal to an appropriate device that increases the rotational velocity of the first agitating element 132, providing for more mixing of the medium. Alternatively, if the agitating element 132 is run at a rotational velocity that creates excessive turbulence and shear forces, leading to cell damage, then such information may be conveyed to a controller of the disposable mixing vessel. The controller may then send a signal to an appropriate device resulting in a rotational velocity decrease of the first agitating element 132. Or, other processing features may be adjusted. For example, if the amount of dissolved oxygen or concentration of nutrient(s) within the liquid is too low, a controller of the disposable mixing vessel that determines that more oxygen or nutrient(s) should be added will send a signal to an appropriate input device for more of the corresponding oxygen or nutrient(s) to be input into the liquid medium.

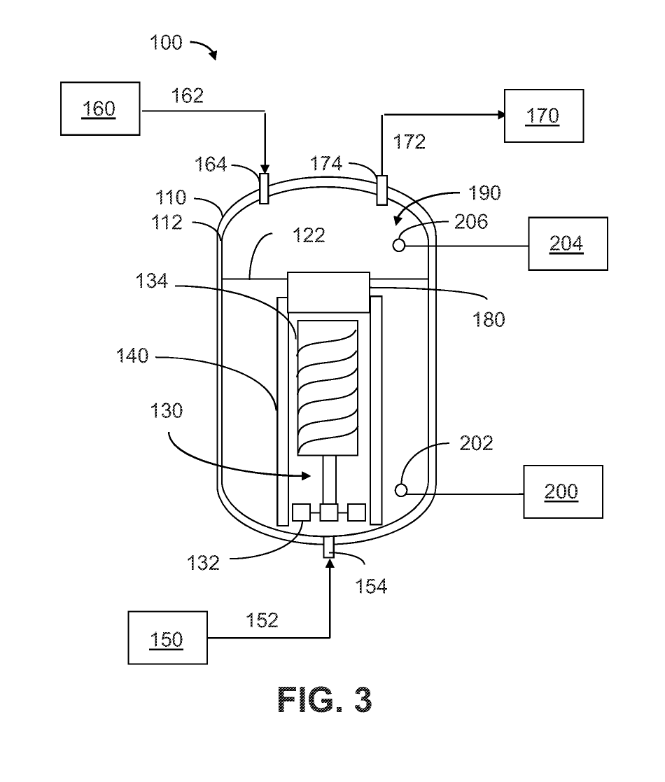

FIG. 3 depicts the disposable mixing vessel having more liquid added to the vessel, resulting in a combined volume of liquid medium 122 reaching a level such that the second agitating element 134 is submerged in the liquid. The added liquid may contain any suitable density of cells, dissolved gas and/or nutrients. As the volume of the liquid medium 122 is increased to reach the level shown in FIG. 3, to cultivate further cell proliferation, although not required, the first agitating element 132 may be operated at a rotational velocity that is greater than the rotational velocity of the first agitating element 132 corresponding to the level of liquid medium in FIG. 2. In some cases, with increasing volumes and cell densities within the medium, it may be preferable to provide progressively greater mixing forces to the liquid medium, so as to cultivate cell proliferation and growth. The second agitating element 134 may also be operated at a rotational velocity that is greater than the previous rotational velocity of the first agitating element 132 of FIG. 2. It can be appreciated that each of the first and second agitating elements 132, 134 may be operated at the same or different rotational velocities. In some embodiments, agitating elements are maintained to operate at relatively low rotational velocities when the level of liquid medium within the disposable mixing vessel is rotational low and are gradually increased as the level of liquid medium rises. In some embodiments, there is a discrete increase in rotational velocities of agitating elements when the liquid medium rises to specified levels within the disposable mixing vessel. As described above, since agitating elements are for providing mixing forces to the liquid medium, it is not necessary for agitating elements to operate in accordance with rotational velocities. The mixing force provided by agitating elements are generally the greatest nearing the end of an exponential growth phase of a cell culture process, generally when the level of liquid medium within the disposable mixing vessel is also at its highest.

Sensor systems 200, 204 of the disposable mixing vessel has probes 202, 206 for monitoring parameters of the liquid medium 122, for example, pH, temperature, dissolved oxygen, dissolved carbon dioxide, osmolality, concentration of nutrient(s) and/or other relevant parameters. An assessment may be made by a controller of the system for dynamic gas control as to the whether the conditions provide suitable viability for cell growth and proliferation. Based on this assessment, the system may provide dynamic gas control of the contents within the disposable mixing vessel to maintain levels of certain parameters within suitable ranges. For example, if it is determined by the system that the liquid medium 122 has an excessive amount of dissolved carbon dioxide that is adverse to cell proliferation and growth, a controller in the system may send an appropriate signal to a device to increase the velocity of the second agitating element 134 such that more of the liquid is directed in an upward flow direction toward the gas/liquid interface on the surface of the liquid medium. In addition, to bring about a decrease in dissolved carbon dioxide within the liquid medium, the system may also cause sweeping gas across the headspace 190 above the surface of the liquid medium to flow at a higher velocity. Accordingly, dissolved carbon dioxide may effectively be stripped from the liquid medium having been continuously and rapidly brought to the surface of the liquid medium where gas-liquid exchange may occur. As such, the carbon dioxide comes out of solution and is swept away into an exhaust system.

By shielding flow of liquid outward from the second agitating element 134 to surrounding outer regions of the vessel upon operation of the second agitating element, the draft tube 140 may be useful for directing flow of the liquid medium upward. In some embodiments, the velocity of upward flow of liquid medium is increased. Directing such upward flow of liquid medium may result in a relatively higher rate of gas exchange than if the draft tube were not present. In some embodiments, vertical baffles 180 are also helpful in maintaining flow of the liquid medium to be vertical in orientation, despite any swirling motions that may be generated by the first and/or second agitating elements 132, 134.

Carbon dioxide removal by interfacial gas exchange between gas in the headspace and gas dissolved at the surface of the liquid medium may be more suitable than conventional carbon dioxide stripping methods such as via sparging. Such gas exchange minimizes shear and bubble damage to cells and may also reduce or eliminate foaming effects.

In addition to gas stripping, rapid gas exchange at the surface of the liquid medium may also be advantageous for enhancing gas dissolution into the liquid. When oxygen demands are high, the system may increase the oxygen composition of sweeping gas in the headspace, resulting in increased transfer of oxygen to the top surface of the circulating liquid. When the requirement for oxygen dissolution is low, the system may reduce oxygen composition of sweeping gas in the headspace and replace the oxygen with another gas, such as air or nitrogen. Variations in oxygen composition of the sweeping gas may have minimal to no impact on the removal rate of carbon dioxide.

The level of dissolved carbon dioxide within the liquid medium can be adjusted or maintained within any desired range. As discussed above, when it is determined by the system that the level of dissolved carbon dioxide in the medium should decrease, the flow rate of sweeping carbon dioxide in the headspace of the disposable mixing vessel can be increased. Increasing the sweeping flow rate of carbon dioxide may more rapidly promote carbon dioxide exchange from the surface of the liquid medium to the headspace and, thus, eliminate carbon dioxide from the medium. The upward flow force generated by an agitating element can also be increased to augment the rate of gas exchange at the surface of the liquid medium. If additional carbon dioxide is required, which may be the case, for example, in early stages of the cell culture process shortly after inoculation within the disposable mixing vessel, carbon dioxide may be added to the sweeping gas mixture in the headspace or, alternatively, may be directly added to the liquid medium via a suitable gas inlet.

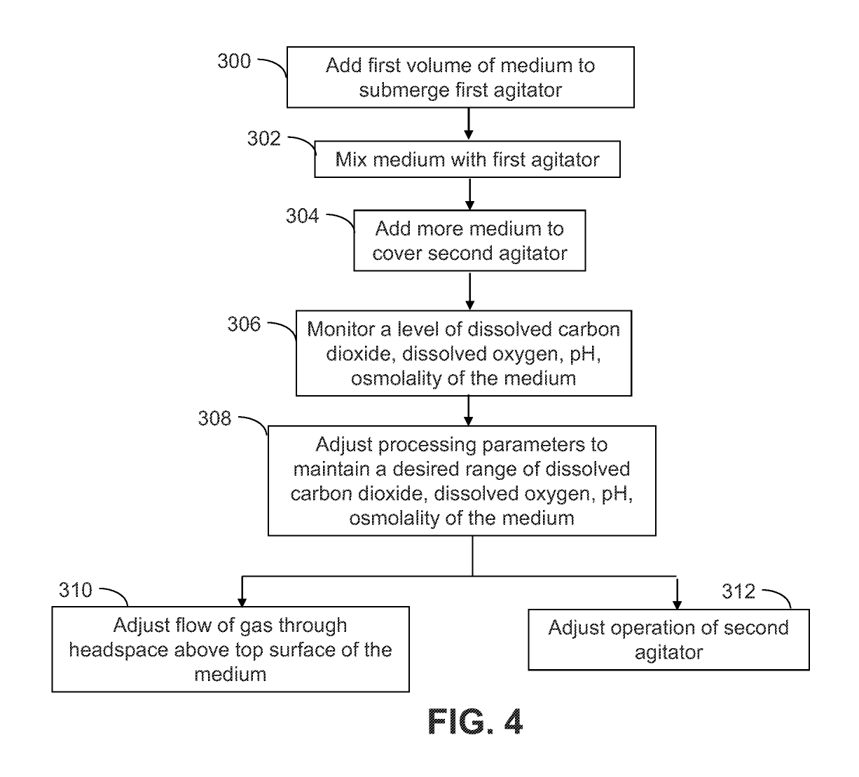

FIG. 4 depicts a flow chart of steps in a procedure for using a system for dynamic gas control to process a liquid medium containing cells in a disposable mixing vessel. In the first step 300, a first volume of liquid medium is added to the disposable mixing vessel to submerge the first agitating element. Although the top surface of the liquid medium reaches above the first agitating element, the top surface does not yet reach the second agitating element. In step 302, the first agitating element is operated to mix the liquid medium so that the cells are exposed to an even distribution of gases and nutrients suitable for cell growth.

In step 304, more liquid medium is added to the disposable mixing vessel so that both the first agitating element and the second agitating element (employed for dynamic gas control) are submerged. Probes and/or sensors associated with the disposable mixing vessel may be used, in step 306, to monitor levels of dissolved carbon dioxide, dissolved oxygen, pH and osmolality of the liquid medium. Based on the monitored levels, in step 308, the system for dynamic gas control may adjust certain processing parameters to maintain levels of dissolved carbon dioxide, dissolved oxygen, pH and osmolality of the liquid medium to be within a desired range. As provided in step 310, one example of a processing parameter that may be adjusted includes the flow of a suitable composition of gas through the headspace above the top surface of the liquid medium. Another example of a processing parameter that may be adjusted is provided in step 312, which includes operation of the second agitating element, such as the rotational velocity. Indeed, the system for dynamic gas control provides for gas flow and agitator mixing forces to be modified in accordance with process demands to improve growth of the cell culture.

In some embodiments, a level of dissolved carbon dioxide within the cell culture medium is maintained to be less than about 10% concentration (e.g., between about 3% and about 5% concentration). The level of dissolved oxygen within the cell culture medium may be maintained to be about 50% concentration or greater. In addition to maintaining desired levels of dissolved oxygen and/or carbon dioxide within a cell culture medium, process control in bioreactors may also focus on maintaining the pH level of the cell culture medium to be between about 6.5 and about 7.5, or between about 7.0 and about 7.4. For example, a buffer solution (e.g., sodium bicarbonate, HEPES, Tricine) may be added to the cell culture medium to maintain the pH of the medium to be within a desired range. To increase the pH of the cell culture medium (e.g., to counteract acidity levels due to increasing amounts of dissolved carbon dioxide), a base solution (e.g., NaOH) may be added. Or, if it is desirable for the pH of the cell culture to be decreased, an acid solution (e.g., HCl) may be added. However, adding a buffer solution or an acid/base solution to the medium may cause the osmolality to increase throughout the cell culture process. High levels of osmolality may incur stress to the cultured cells, negatively impacting yield and productivity. The level of osmolality within the cell culture medium may be maintained to be less than about 600 mOsmol/kg (e.g., between about 300 and about 560 mOsmol/kg, or between about 400 and about 500 mOsmol/kg), or less than about 400 mOsmol/kg from the beginning of the growth phase to the end of the production phase of the cell culture process.

Another source of stress to the cultured cells in a bioreactor may come from bubbles delivered to the liquid via a sparger. In some cases, gas bubbles introduced from a sparger and the breakage of those bubbles near cells can incur damage to sensitive cells and can cause cell death. Sparged gases may also create excess foam. Antifoam agents have conventionally been used to mitigate excessive foam, which may interfere with preferred gas exchange mechanisms and may give rise to downstream purification issues.

The combined effects resulting from the above described sources of stress include lower rates of cell growth, longer batch times, decreased productivity and yield, lower viability, increased cell lysis, more difficult process development and scale-up and degradation of protein products (e.g., by proteolytic enzymes released from bursting cells). The contents of bursting cells can also add to purification issues, particularly if antifoam agents must be employed in the process.

The stresses listed above are generally mitigated or abolished by systems and methods described herein for incorporation in disposable mixing vessels. Using such systems and methods may help to increase yield and productivity, facilitate process development and scale-up, improve product quality and simplify purification.

When systems and methods described are implemented for disposable bioreactors or disposable fermentors, which have high cell/organism batch densities, oxygen consumption and/or power requirements may be reduced. In some embodiments, for high density batches of cells/organisms typically found in disposable bioreactors or disposable fermentors, high levels are recorded for oxygen transfer efficiency, effectively reducing either or both oxygen consumption and/or power requirements.

For purposes described herein, oxygen transfer efficiency (OTE) is measured as the amount of oxygen consumed in a vessel (e.g., reactor or fermentor) compared to the oxygen input into the vessel. For example, a 25% measure of air OTE would indicate that a quarter of the oxygen available through input of air into the vessel had been consumed by the process (e.g., reaction or fermentation) occurring within the vessel. For instance, the OTE for disposable bioreactors may be increased to 33% or more when embodiments described herein are used to employ dynamic gas control in a disposable mixing vessel.

Various gas parameters were measured for a disposable bioreactor having a system for dynamic gas control installed therein as compared with a disposable bioreactor without the system for dynamic gas control.

In a Conventional Example, a fermentation broth was mixed in a disposable fermentor. Air was injected at an inner mixing region from the bottom of the fermentor directly toward the agitation system, and oxygen-rich gas was injected outside of the inner mixing region via a full-ring sparger.

In the Example, the disposable bioreactor incorporates a second agitating element that generates upward flow currents and is located above the first agitating element, similar to the embodiment depicted in FIGS. 1-3. Thus, liquid medium was added to the disposable bioreactor to submerge the first agitating element. The first agitating element was operated to mix the medium according to a suitable process for cell culture. Additional liquid medium was then added to the disposable bioreactor to fully submerge the second agitating element in the liquid medium. Operating the second agitating element and also inlet/outlet ports to sweep gas compositions across the headspace above the liquid medium, the system for dynamic gas control is employed to maintain the level of dissolved oxygen to be at least 50%, the level of dissolved carbon dioxide to be less than 10%, the pH to be between 6.5 and 7.5 and the level of osmolality to be less than 400 mOsmol/kg. The second agitating element was operated at a rotational velocity greater than the rotational velocity which the first agitating element was operated at prior to adding the second volume of liquid medium to the disposable bioreactor. A number of measured results for this example as compared with a conventional example are provided in Table 1 below.

TABLE 1

| | Conventional | | |
|---|---|---|---|
| | Example | Example | |
| Working Volume (L) | 200 | 200 | |
| O₂ sparge to headspace (slpm) | 1.2 | 0.006 | average |
| O₂ to headspace (slpm) | 0 | 5.7 | average |
| Air to headspace (slpm) | Not measured | 0.3 | average |

| | | |
|---|---|---|
| O₂ transfer rate (mmol O₂/L/h) | 4 | 4 |
| Overall OTE (%) | 25.9% | 33.6% |

Using a system for dynamic gas control to maintain characteristics of the liquid medium within a disposable bioreactor may result in a higher OTE than the OTE that would have been obtained if the system for dynamic gas control had not been installed in the disposable bioreactor. As shown in the Table, the overall OTE for the Example was greater compared with the Conventional Example. Additionally, though not explicitly measured, power consumption requirements for embodiments incorporating systems and methods of dynamic gas control described herein in conjunction with processes for cell culture may be less than power consumption requirements for conventional systems and methods.

From the foregoing, it should be appreciated that the present invention thus provides various methods and systems for controlling the dissolved carbon dioxide level, dissolved oxygen level, pH and osmolality during a mammalian cell culture process to enhance cell viability and biologic product yield. Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the scope of the invention. Accordingly, the foregoing description and drawings are by way of example only.

What is claimed is:

1. A method of processing a liquid in a disposable mixing vessel, the method comprising:
providing the disposable mixing vessel having a first agitating element located at a lower mixing region of the disposable mixing vessel;
providing a system for dynamic gas control within the disposable mixing vessel employing a second agitating element located above the first agitating element;
adding a first volume of liquid to the disposable mixing vessel having a top surface height that is lower than the second agitating element within the disposable mixing vessel;
providing living organisms in the first volume of liquid;
operating the first agitating element to mix the first volume of liquid within the disposable mixing vessel and to help provide a suitable living environment for the living organisms;
subsequent to operating the first agitating element, adding a second volume of liquid to the first volume of liquid within the disposable mixing vessel resulting in a combined volume of liquid that has a top surface height that is higher than the second agitating element;
operating the second agitating element to mix the combined volume of liquid within the disposable mixing vessel and to help provide a suitable living environment for the living organisms; and
operating the system for dynamic gas control for maintaining at least one of a range of dissolved carbon dioxide, a range of dissolved oxygen, a range of pH or a range of osmolality within the combined volume of liquid.

2. The method of processing a liquid of claim 1, wherein operating the second agitating element comprises directing flow of a portion of the combined volume of liquid upward within the disposable mixing vessel.

3. The method of processing a liquid of claim 1, wherein operating the system for dynamic gas control comprises sensing a level of at least one of dissolved carbon dioxide, dissolved oxygen, pH or osmolality within the combined volume of liquid.

4. The method of processing a liquid of claim 3, wherein operating the system for dynamic gas control comprises flowing gas through a headspace above a top surface of the liquid.

5. The method of processing a liquid of claim 4, wherein flowing gas through the headspace above the top surface of the liquid comprises introducing the gas into the disposable mixing vessel and removing the gas from the disposable mixing vessel.

6. The method of processing a liquid of claim 4, wherein flowing gas through the headspace above the top surface of the liquid comprises flowing at least one of oxygen, carbon dioxide, nitrogen, air or combinations thereof within the disposable mixing vessel.

7. The method of processing a liquid of claim 4, further comprising adjusting a characteristic of gas flow through the headspace above the top surface of the liquid based on the sensed level of at least one of dissolved carbon dioxide, dissolved oxygen, pH or osmolality within the liquid.

8. The method of processing a liquid of claim 3, further comprising adjusting an operating level of the second agitating element based on the sensed level of at least one of dissolved carbon dioxide, dissolved oxygen, pH or osmolality within the liquid.

9. The method of processing a liquid of claim 1, wherein operating the system for dynamic gas control comprises decreasing a level of dissolved carbon dioxide or a level of osmolality within the liquid.

10. The method of processing a liquid of claim 1, wherein operating the system for dynamic gas control comprises maintaining a pH within the liquid to be between about 6.5 and about 7.5.

11. The method of processing a liquid of claim 1, wherein operating the system for dynamic gas control comprises maintaining a level of dissolved carbon dioxide within the liquid to be less than about 10% concentration.

12. The method of processing a liquid of claim 1, wherein operating the system for dynamic gas control comprises maintaining a level of dissolved oxygen within the liquid to be greater than about 50% concentration.

13. The method of processing a liquid of claim 1, wherein operating the system for dynamic gas control comprises maintaining a level of osmolality within the liquid to be less than about 400 mOsmol/kg.

14. A system for processing a liquid, the system comprising:
a disposable mixing vessel constructed and arranged to contain the liquid;
a first agitating element located at a lower mixing region of the disposable mixing vessel;
a second agitating element located above the first agitating element and constructed and arranged to direct flow of liquid upward within the disposable mixing vessel;
a gas inlet port disposed at an upper region of the disposable mixing vessel adapted to provide entry of a gas into a headspace above the top surface of the liquid;
a gas outlet port disposed at the upper region of the disposable mixing vessel adapted to permit exit of the gas from the headspace above the top surface of the liquid;
at least one sensor for sensing at least one of a level of dissolved carbon dioxide, a level of dissolved oxygen, a pH or a level of osmolality of the liquid; and
a controller configured to control operation of the first and second agitating elements, the gas inlet and outlet ports and the at least one sensor in two operating modes including a first mode in which a liquid level in the vessel is above the first agitating element and below the second agitating element, and a second mode in which the liquid level in the vessel is above the second agitating element, the first mode including controlling the first agitating element to mix the liquid in the vessel at a speed suitable for living organisms in a condition with the liquid level below the second agitating element, and the second mode including controlling a speed of the second agitating element or a characteristic of gas flow through the headspace above the top surface of the liquid based on a sensed parameter of the at least one of the level of dissolved carbon dioxide, the level of dissolved oxygen, the pH or the level of osmolality of the liquid so as to maintain at least one of a range of dissolved carbon dioxide, a range of dissolved oxygen, a range of pH or a range of osmolality within the liquid.

15. The system for processing a liquid of claim 14, wherein the first agitating element comprises at least one impeller.

16. The system for processing a liquid of claim 15, wherein the at least one impeller comprises a Ruston turbine or a pitched blade turbine.

17. The system for processing a liquid of claim 14, further comprising a shaft coupling the first agitating element and the second agitating element together.

18. The system for processing a liquid of claim 14, wherein the disposable mixing vessel comprises a disposable bioreactor or a disposable fermentor.

19. The system for processing a liquid of claim 18, further comprising a support structure for the disposable mixing vessel, wherein the disposable bioreactor or disposable fermentor comprises a disposable bag lining an inner wall of a support structure.
